(19) United States
(12) Patent Application Publication
(10) Pub. No.: US 2025/0018215 A1
(43) Pub. Date: Jan. 16, 2025

(54) LIGHT THERAPY DEVICE

(71) Applicant: SHENZHEN KAIYAN MEDICAL EQUIPMENT CO., LTD, Shenzhen (CN)

(72) Inventors: ALAIN DIJKSTRA, Amstelveen (NL); ZENG XIANQI, Shenzhen (CN)

(73) Assignee: SHENZHEN KAIYAN MEDICAL EQUIPMENT CO., LTD, Shenzhen (CN)

(21) Appl. No.: 18/679,503

(22) Filed: May 31, 2024

(30) Foreign Application Priority Data
Jul. 14, 2023 (CN) 2023218481617

(51) Int. Cl. A61N 5/06; A61N 5/067

(52) U.S. Cl. CPC A61N 5/0613; A61N 5/067; A61N 2005/0632; A61N 2005/0645; A61N 2005/0651; A61N 2005/0659; A61N 2005/0661; A61N 2005/0663

(57) ABSTRACT

A light therapy device comprises a flexible casing made with flexible silicone material. A Flexible Printed Circuit Board (FPCB) is installed over a front portion of the flexible casing. The FPCB comprises a plurality of stimulation elements and at least one pair of primary electrodes for therapeutic and recreational purpose. A detachable controller is attached over a back portion of the flexible casing. The controller comprises at least one pair of secondary electrodes, a rechargeable battery, and a user interface. The at least one pair of secondary electrodes engages with the at least one pair of primary electrodes to transmit the power from the rechargeable battery to the FPCB. Further, a flexible diaphanous sheet mounted over the FPCB through which the light therapy is provided to a user.

### FIELD OF THE INVENTION

The present invention generally relates to the domain of therapeutic and recreational devices. More specifically, the present invention relates to devices that can stick to an individual's skin to provide various kinds of stimulation, including irradiation, heating, cooling, and vibrations.

### BACKGROUND OF THE INVENTION

Various therapeutic devices/patches have been developed for therapeutic and recreational purposes for quite some time. These devices utilize electrodes, heating elements, cooling mechanisms, and irradiation sources like LEDs and lasers to deliver energy to specific body areas. While commonly employed for pain relief, specific therapy devices serve the purpose of facilitating wound healing. Traditional bandages or patches are conventionally designed for single use, thereby exacerbating the issue of medical waste. Furthermore, conventional patches frequently induce discomfort when detached from the user's body. Furthermore, traditional patches, when adhered to the user's skin, may fail to deliver effective therapy due to the uneven shape of the skin, such as in the area below the eyes. Addressing these described challenges, the present invention introduces an innovative approach to light therapy devices to minimize medical waste and enhance user comfort during use and removal.

US20160015962A1 discloses a flexible patch that emits light in the UV, visible, and/or infrared electromagnetic spectrums. The patch includes a feedback process and system using a plurality of sensors and a controller on the patch to accelerate the wound healing process by providing adaptable, controlled light exposure and electrical stimulation. The patch also monitors the healing process for signs of infection and eliminates bacterial infections by sanitizing the infected site and transforming the information through wireless mode to a central location for storage and interpretation by a physician. The disclosed patch also receives feedback from the physician and provides the same to the user. In addition, the physician from the remote location can also instruct the user on how to operate.

The prior art, as exemplified by U.S. Pat. No. US'962 reveals a flexible therapy patch designed for administering therapeutic sessions to aid in the healing of wounds on a user's body. The disclosed patch in the above prior art facilitates communication between the user and a physician by exchanging information and provides continuous monitoring of the healing process while eliminating bacteria or fungi from the wound. Despite these commendable features, a notable limitation of the prior art is its non-reusability. Moreover, the uneven shape of certain body areas, such as the region below the eye, poses a challenge during patch usage, as the adhesive tape alone may not suffice to cover the entire irregular surface. Building upon this prior art, the present invention introduces improvements to address these shortcomings, including reusability and enhanced adaptability to uneven body contours.

US20220226668A1 discloses an LED patch for skin care. The LED patch includes a substrate part, a circuit pattern part formed in at least two layers on the substrate part, a light source part including one or more LEDs mounted on one surface of the substrate part, and a cover part configured to cover at least one surface of the substrate part. The disclosed LED patch is utilized to provide a therapeutic session to a user as per the condition/desire of the user.

The above prior art, US'668, disclosed an LED patch helpful in skin care. The LED patch includes a cover part, which makes the LED patch moisture-free. The cover part is made of silicon material, providing comfort to the user using the LED patch. However, the re-usage of the LED patch is not disclosed. Also, the design of the disclosed LED patch is complicated.

Therefore, there is a need in the art for light therapy-based devices that do not suffer from the aforementioned deficiencies.

### OBJECTS OF THE INVENTION

Some of the objects of the invention are as follows:

An object of the present invention is to develop a device designed to administer light therapy to a specific body area, aiming to assist in alleviating pain for the user.

Another object of the present invention is to develop a device that minimizes medical waste.

Another object of the present invention is to develop a device that ensures the user does not experience discomfort while using the device.

Another object of the present invention is to develop a device that can be used multiple times, promoting reusability.

Another object of the present invention is to develop a device that is rechargeable, enhancing the longevity and sustainability of the device.

Another object of the present invention is to develop a water-resistant device, enhancing the durability and usability of the device in various conditions.

Another object of the present invention is to develop a lightweight device, aiming to provide comfort and convenience to the user during usage.

Yet another object of the present invention is to develop a device that is capable of emitting various lights, including blue light, red light, etc.

### SUMMARY OF THE INVENTION

According to an embodiment of the present invention, there is provided a light therapy device. The proposed device comprises a patch and a detachable controller. Further, the patch comprises a flexible casing made with silicone material. The flexible casing further comprises a front portion, a back portion, and a peripheral wall. The peripheral wall is crafted over the periphery of the front portion. The patch further includes a plurality of protrusions of a predetermined length crafted over the flexible casing. Further, a Flexible Printed Circuit Board (FPCB) installed over the front portion of the flexible casing. The Flexible Printed Circuit Board (FPCB) comprises a plurality of stimulation elements. The patch further includes a flexible diaphanous sheet made with liquid silicone material and is arranged over the Flexible Printed Circuit Board (FPCB). Moreover, a plurality of cuts crafted over the periphery of the Flexible Printed Circuit Board (FPCB) at a predetermined distance between each of the cuts. The number of cuts is equivalent to the number of protrusions. Further, each of the cuts is engaged with a corresponding protrusion. Furthermore, at least one deformable element is inbuilt within the flexible casing.

In one embodiment of the invention, at least one pair of through-holes is formed over the back portion of the flexible casing.

In one embodiment of the invention, the flexible casing is constructed from opaque material to obstruct the passage of light through the flexible casing.

In one embodiment of the invention, the Flexible Printed Circuit Board (FPCB) comprises at least one pair of primary electrodes passing through the at least one pair of through-holes.

In one embodiment of the invention, the detachable controller is attached to the back portion of the flexible casing. The detachable controller comprises a hollow housing that includes at least one pair of secondary electrodes, the at least one pair of secondary electrodes magnetically attached to the at least one pair of primary electrodes. Further, a rechargeable battery is installed within the controller to power the light therapy device. Moreover, a user interface is crafted over the hollow housing and is electrically connected to the rechargeable battery to operate the light therapy device.

In one embodiment of the invention, the user interface includes but is not limited to a push button and a touch panel.

In one embodiment of the invention, the shape of the patch includes but is not limited to a star shape, a moon shape, a hexagon shape, a heptagon shape, an octagon shape, and a heart shape.

In one embodiment of the invention, the plurality of stimulation elements is selected from a group consisting of irradiation sources, heating elements, cooling elements, vibration elements, ultrasonic wave generators, electrodes, and combinations thereof.

In one embodiment of the invention, the irradiation sources are configured to emit electromagnetic radiation in a wavelength range of 300 nm to 1200 nm.

In one embodiment of the invention, the light therapy device is detachably attached to a user's skin using an adhesive agent to enable a user to reuse the light therapy device multiple times.

In one embodiment of the invention, the at least one deformable element is made with a metallic material.

In one embodiment of the invention, an insulation separates the Flexible Printed Circuit Board (FPCB) and the primary electrodes from the deformable element.

In one embodiment of the invention, the adhesive agent includes but is not limited to a glue, a gel, and a double-sided tape.

In one embodiment of the invention, the flexible diaphanous sheet is sealed with the Flexible Printed Circuit Board (FPCB) via an encapsulating glue.

In one embodiment of the invention, the flexible diaphanous sheet is skin-friendly. In one embodiment of the invention, the light therapy device is water resistant.

### BRIEF DESCRIPTION OF DRAWINGS

The accompanying drawings illustrate the best mode for carrying out the invention as presently contemplated and set forth hereinafter. The present invention may be more clearly understood from a consideration of the following detailed description of the preferred embodiments taken in conjunction with the accompanying drawings wherein like reference letters and numerals indicate the corresponding parts in various figures in the accompanying drawings, and in which:

FIG. 1A illustrates a perspective view of a light therapy device in which a detachable controller is detached from a patch, in accordance with an embodiment of the present invention;

FIG. 1B illustrates a perspective view of the light therapy device in which the detachable controller is assembled with the patch, in accordance with an embodiment of the present invention;

FIG. 2A illustrates an exploded view of the patch, in accordance with an embodiment of the present invention;

FIG. 2B illustrates an exploded view of a star shape patch of FIG. 2A;

FIG. 3 illustrates an exploded view of the detachable controller, in accordance with an embodiment of the present invention;

FIG. 4A illustrates an exploded view of the patch designed with a deformable element, in accordance with an embodiment of the present invention;

FIG. 4B illustrates an exploded view of the patch designed with the deformable element in another configuration, in accordance with an embodiment of the present invention;

FIG. 5A illustrates a bottom view of the light therapy device in various shapes, in accordance with an embodiment of the present invention; and

FIG. 5B illustrates a top view of the light therapy device in various shapes of FIG. 5A, in accordance with an embodiment of the present invention.

### DETAILED DESCRIPTION OF THE INVENTION

Embodiments of the present invention disclosure will be described more fully hereinafter with reference to the accompanying drawings in which like numerals represent like elements throughout the figures, and in which example embodiments are shown.

The detailed description and the accompanying drawings illustrate the specific exemplary embodiments by which the disclosure may be practiced. These embodiments are described in detail to enable those skilled in the art to practice the invention illustrated in the disclosure. It is to be understood that other embodiments may be utilized, and other changes may be made, without departing from the spirit or scope of the present disclosure. The following detailed description is, therefore, not to be taken in a limiting sense, and the scope of the present invention disclosure is defined by the appended claims. Embodiments of the claims may, however, be embodied in many different forms and should not be construed as limited to the embodiments set forth herein.

In the context of this specification, terms like “light” and “illumination”, etc. refer to electromagnetic radiation in wavelength ranges varying from the visible light wavelengths (380-750 nm) to Infrared (IR) wavelengths (750 nm-1400 nm), wherein the range is inclusive of visible light and IR wavelengths. The IR radiation may also be categorized into several categories according to respective wavelength ranges, which are again envisaged to be within the scope of this invention. A commonly used subdivision scheme for IR radiation includes Near IR (0.75-1.4 μm), Short-Wavelength IR (1.4-3 μm), Mid-Wavelength IR (3-8 μm), Long-Wavelength IR (8-15 μm) and Far IR (15-1000 μm).

In the context of the specification, “Light Emitting Diodes (LEDs)” are envisaged to be semiconductor devices that emit electromagnetic radiation when current is applied through them. LEDs are characterized by their superior power efficiencies, smaller sizes, rapidity in switching, physical robustness, and longevity when compared with incandescent or fluorescent lamps. In that regard, the plurality of LEDs may be through-hole type LEDs (generally used to produce electromagnetic radiations of red, green, yellow, blue, and white colors), Surface Mount LEDs, Bi-color LEDs, Pulse Width Modulated RGB (Red-Green-Blue) LEDs, and high-power LEDs, etc.

Materials used in the construction of LEDs may vary from one embodiment to another depending upon the frequency of radiation required. Different frequencies can be obtained from LEDs made from pure or doped semiconductor materials. Commonly used semiconductor materials include nitrides of Silicon, Gallium, Aluminum, Boron, Zinc, Selenide, etc., in pure form or doped with elements such as Aluminum and Indium, etc. For example, red and amber colors are produced from Aluminum Indium Gallium Phosphide (AlGaInP) based compositions, while blue, green, and cyan use Indium Gallium Nitride based compositions. White light may be produced by mixing red, green, and blue lights in equal proportions, while varying proportions may be used to generate a wider color gamut. White and other colored lightings may also be produced using phosphor coatings such as Yttrium Aluminum Garnet (YAG) in combination with a blue LED to generate white light and Magnesium doped potassium fluorosilicate in combination with a blue LED to generate red light. Additionally, near Ultraviolet (UV) LEDs may be combined with europium-based phosphors to generate red and blue lights and copper and zinc-doped zinc sulfide-based phosphors to generate green light.

In addition to conventional mineral-based LEDs, one or more LEDs may also be provided on an organic LED (OLED) flexible panel or an inorganic LED-based flexible panel. Such OLED panels may be generated by depositing organic semiconducting materials over Thin Film Transistor (TFT) based substrates. Further, discussion on generation of OLED panels can be found in Bardsley, J. N (2004), “International OLED Technology Roadmap”, IEEE Journal of Selected Topics in Quantum Electronics, Vol. 10, No. 1, that is included herein in its entirety, by reference. An exemplary description of flexible inorganic light-emitting diode strips can be found in granted U.S. Pat. No. 7,476,557 B2, titled “Roll-to-roll fabricated light sheet and encapsulated semiconductor circuit devices”, which is included herein in its entirety, by reference.

In several embodiments, the one or more LEDs may also be micro-LEDs described through U.S. Pat. Nos. 8,809,126 B2, 8,846,457 B2, 8,852,467 B2, 8,415,879 B2,8,877,101 B2, 9,018,833 B2 and their respective family members, assigned to NthDegree Technologies Worldwide Inc., which are included herein by reference, in their entirety. The one or more LEDs, in that regard, may be provided as a printable composition of the micro-LEDs, printed on a substrate.

In the context of the specification, the phrase “diaphanous material” refers to a material that allows at least a portion of one or more forms of electromagnetic radiation (such as Infrared, Ultraviolet, X-rays, Visible Light, Microwaves, Radio Waves, etc.) to pass through them. The diaphanous materials can be transparent (allowing one or more forms of electromagnetic radiation to pass through with minimal scattering) or translucent (allowing one or more forms of electromagnetic radiation to pass through with appreciable diffusion or scattering). Diaphanous materials can be dense, like glass, or have an open structure, like wire mesh or a woven fabric.

The proposed therapeutic light therapy device is designed to administer a healing session to the user. The light therapy device comprises a patch and a detachable controller. The detachable controller includes a hollow housing, a user interface, and a rechargeable battery. In addition, at least one pair of secondary electrodes is crafted over a bottom portion of the hollow housing of the detachable controller.

Further, the patch is constructed with a flexible casing made of silicone material and is available in various shapes, such as stars, moons, hexagons, heptagons, octagons, and hearts. The flexible casing comprises a front portion, a back portion, and a peripheral wall. The front portion is surrounded by a Flexible Printed Circuit Board (FPCB) that comprises multiple stimulation elements. The stimulation elements include but are not limited to a plurality of Light Emitting Diodes (LEDs), a plurality of lasers, a plurality of tensed electrotherapy elements, and a plurality of heating elements. The stimulation elements are responsible for emitting diverse colors of light/heat/electrotherapy to provide therapy to the user. Further, the Flexible Printed Circuit Board (FPCB) also includes at least one pair of primary electrodes, which are magnetically connected to the at least one pair of secondary electrodes to transmit the power from the rechargeable battery to the Flexible Printed Circuit Board (FPCB).

In addition, the periphery of the Flexible Printed Circuit Board (FPCB) is designed with a plurality of cuts. On the other hand, a plurality of protrusions protruded on the flexible casing. The number of the plurality of cuts is equivalent to the number of the plurality of protrusions. Each of the cuts engages with each of the corresponding protrusions to hold the FPCB strongly and securely within the patch. In addition, a flexible diaphanous sheet lay over the Flexible Printed Circuit Board (FPCB). The flexible diaphanous sheet is made of a liquid silicone material. While manufacturing, there are chances that the liquid silicone may enter between the Flexible Printed Circuit Board (FPCB) and the flexible casing. The engagement of the cuts with the protrusions restricts the liquid silicone from entering between the FPCB and the flexible casing. Further, the flexible diaphanous sheet herein allows the emitted light to pass through the flexible diaphanous sheet and deliver therapeutic benefits to the user.

Further, to ensure a secure grip between the user's skin and the light therapy device, a deformable element, which may be a metallic wire or sheet or grid of metallic wire/sheet, is integrated into the flexible casing. Furthermore, an adhesive agent is detachably attached to the patch to affix the light therapy device over the user's body portion for starting the therapeutic session. The adhesive agent for attaching the patch over the user's skin is skin-friendly and makes the device used multiple times. Some of the examples of skin-friendly adhesive agents may include, but are not limited to, silicones polydimethylsiloxane (PDMS) and silicone acrylates, acrylics, hydrocolloids, cyanoacrylates, etc. Further, the adhesive agent can be in the form of a glue, a gel, or double-sided tape. The material of the adhesive agent would be diaphanous.

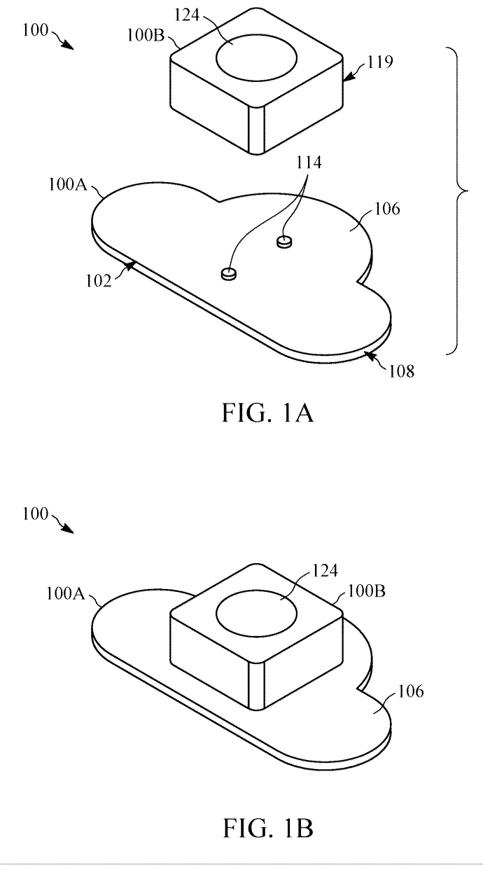

Several embodiments of the present invention will now be elucidated with the help of FIG. 1A-5B.

FIG. 1A illustrates a perspective view of a light therapy device 100 (referred to as “the device 100”) in which a detachable controller 100B is detached from a patch 100A, in accordance with an embodiment of the present invention. In several embodiments of the present invention, the light therapy device 100 comprises a patch 100A and a detachable controller 100B. The detachable controller 100B is utilized to control the patch 100A through numerous operations, such as switching on/off the device 100, and changing the lights to provide a therapeutic session to a user.

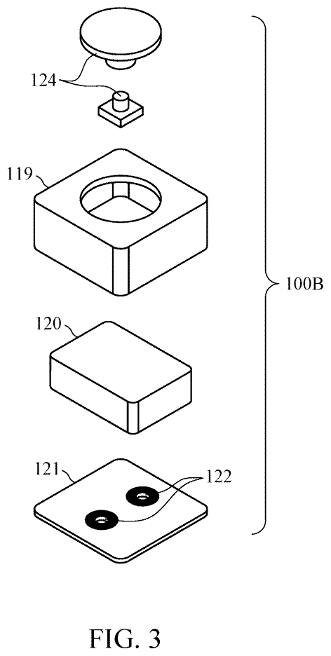

The detachable controller 100B is comprised of a hollow housing 119, a user interface 124, a rechargeable battery (as shown in FIG. 3), and at least one pair of secondary electrodes (as shown in FIG. 3). The user interface 124 may include but is not limited to a push button and a touch panel. The user interface 124 located on the top surface of the controller 100B enabling the user to operate various functions, such as switching the lights or other different modes of operation. Alternatively the user interface 124 can be situated on the side walls of the controller 100B.

In several embodiments of the present invention, the patch 100A comprises a flexible casing 102. The flexible casing 102 is made of a silicone material. The choice of silicone material is deliberate, selected due to the inherent properties such as flexibility, durability, and biocompatibility. The flexibility of the silicone material allows the patch 100A to conform to diverse shapes and contours, ensuring comfort and adaptability to different body surfaces.

In addition, the durable nature of the silicone material contributes to the prolonged effectiveness of the light therapy device 100. Furthermore, the biocompatible properties of silicone, coupled with the electrical insulation capability, mitigate the chances of injuries related to electrical short circuits. Moreover, the flexible casing 102 renders the patch 100A waterproof and resistant to temperature variations.

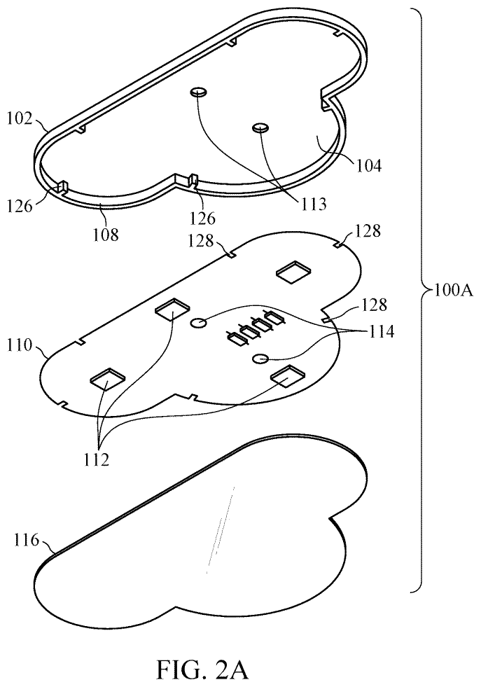

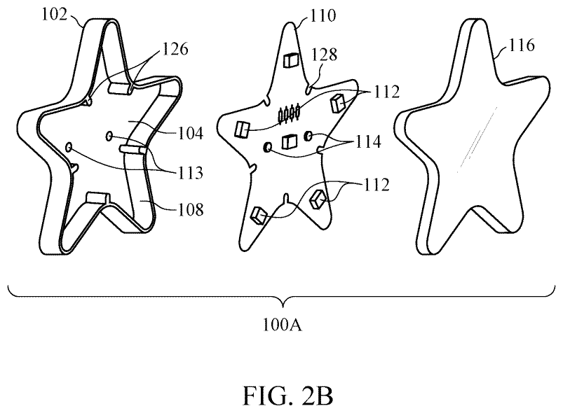

In several embodiments of the present invention, the flexible casing 102 comprises a front portion (104, as shown in FIG. 2A), a back portion 106, and a peripheral wall 108. The peripheral wall 108 is meticulously crafted over the front portion (104, as shown in FIG. 2A). The patch 100A further includes a Flexible Printed Circuit Board (FPCB) (as shown in FIG. 2A), which surrounds the flexible casing 102 from the side of the front portion (104, as shown in FIG. 2A). The Flexible Printed Circuit Board (FPCB) (as shown in FIG. 2A) comprises of a plurality of stimulation elements (as shown in FIG. 2A) and at least one pair of primary electrodes 114. The at least one pair of primary electrodes 114 is utilized to transmit the power from the rechargeable battery (as shown in FIG. 3) to the Flexible Printed Circuit Board (FPCB) (as shown in FIG. 2A).

FIG. 1B illustrates a perspective view of the light therapy device 100, in which the detachable controller 100B is assembled with the patch 100A, in accordance with an embodiment of the present invention. In several embodiments of the present invention, the detachable controller 100B is securely attached to the patch 100A. The at least one pair of primary electrodes 114 is magnetically engaged with the at least one pair of secondary electrodes (as shown in FIG. 3) to transmit the power from the rechargeable battery (as shown in FIG. 3) to the Flexible Printed Circuit Board (FPCB) (as shown in FIG. 2A).

FIG. 2A and FIG. 2B illustrate an exploded view of the patch 100A, and an exploded view of a star-shaped patch 100A, respectively, in accordance with an embodiment of the present invention. In several embodiments of the present invention, the star shape patch 100A has the same configuration as of the patch 100A in FIG. 2A, only the shape of the patch 100A is different.

In several embodiments of the present invention, the patch 100A includes the flexible casing 102, the Flexible Printed Circuit Board (FPCB) 110, and a flexible diaphanous sheet 116 made of a diaphanous material. The diaphanous material may be a Polycarbonate, PMMA or Acrylic, Polyethylene Terephthalate (PET), Amorphous Co-polyester (PETG), Polyvinyl Chloride (PVC), Liquid Silicone Rubber (LSR), Cyclic Olefin Copolymers, Polyethylene (PE), Polystyrene (PS), Thermoplastic polyurethanes (TPU), Polyvinyl butyral (PVB), Co-polymer ethylene vinyl acetate (EVA).

Moreover, the flexible casing 102 is crafted with at least one pair of through-holes 113. The through-holes 113 are utilized for passing the at least one pair of primary electrodes 114 from the flexible casing 102 to magnetically attach the at least one pair of primary electrodes 114 with the at least one pair of secondary electrodes (as shown in FIG. 3).

In several embodiments of the present invention, the peripheral wall 108 is crafted along the periphery of the flexible casing 102, featuring a plurality of protrusions 126 of predetermined length. The plurality of protrusions 126 is strategically oriented towards the center of the flexible casing 102, although it is recognized that alternate configurations may be conceivable by those skilled in the art.

In several embodiments of the present invention, a plurality of cuts 128 is crafted over the periphery of the Flexible Printed Circuit Board (FPCB) 110. The plurality of cuts 128 is engaged with the plurality of the protrusions 126 to make a strong grip in between the Flexible Printed Circuit Board (FPCB) 110 and the flexible casing 102.

In several embodiments of the present invention, the Flexible Printed Circuit Board (FPCB) 110 incorporates the plurality of stimulation elements 112 and the at least one pair of primary electrodes 114. The plurality of stimulation elements 112 may be selected from a group consisting of irradiation sources, heating elements, cooling elements, vibration elements, ultrasonic wave generators, electrodes, and combinations thereof. Furthermore, irradiation sources may be selected from a group consisting of Light Emitting Diodes (LEDs), and lasers. Additionally, the irradiation sources may be configured to operate in one or more of a pulse mode and a continuous mode. In the case of a stimulation element being an irradiation source, the stimulation element would be emitting electromagnetic radiation. The wavelengths of emitted light from the irradiation sources fall within the ranges of 300-1200 nm.

In the case of a stimulation element being a vibration element, the stimulation element would be a vibrating head connected to an eccentric mass rotating motor or a linear resonant motor. In case of a stimulation element being a heating element, the stimulation element may be selected from a group consisting of metal heating elements, ceramic heating elements, semiconductor heating elements, thick film heating elements, polymer-based heating elements, composite heating elements, and combination heating elements. In the case of a stimulation element being a cooling element, the stimulation element may be a thermoelectric cooler, also known as a Peltier heat pump. In the case of a stimulation element being an ultrasonic wave generator, a wave generation head may consist of a quartz crystal fused with a metal plate. The quartz crystal may produce ultrasonic waves due to the piezoelectric effect. Ultrasonic wave therapy may be used in applications such as treatment of chronic pain, improvement in blood circulation, and tissue repair.

In the case of a stimulation element being an electrode, the stimulation element may be embodied as an open-ended conductor. The electrode may then be able to provide Transcutaneous Electrical Nerve Stimulation (TENS), Electronic Muscle Stimulation (EMS), and Microcurrent Electrical Therapy (MET) to the body of a user. TENS therapy uses low-voltage currents to provide pain relief. Electrical impulses are delivered through electrodes placed on the surface of the body of the user. The electrodes are placed at or near nerves where the pain is located or at certain known trigger points. EMS therapy is similar to TENS therapy, the difference being that EMS is applied to key muscle groups instead of a generalized application. The electrical signals in EMS cause certain muscles to undergo contractions and tightening. Moreover, electrical impulses in EMS are stronger when compared with TENS therapy. MET in contrast uses a current of amplitude less than 1 milliampere and a frequency of 0.5 Hz and is indicated for the treatment of pain.

Further, in several embodiments, the flexible diaphanous sheet 116 is mounted over the Flexible Printed Circuit Board (FPCB) 110. The flexible diaphanous sheet 116 is securely attached to the Flexible Printed Circuit Board (FPCB) 110 via encapsulating glue, ensuring a robust connection. The flexible diaphanous sheet 116, made of silicone material, shares properties identical to those of the silicone material of the flexible casing 102. In addition, the silicone material of the flexible diaphanous sheet 116 is translucent. The translucent nature of the sheet facilitates the passage of light without compromising intensity or wavelength effectiveness. Additionally, the silicone material imparts a soft texture to enhance user comfort during the application of the light therapy device 100.

In an alternative embodiment, a set of electrodes, which may be the primary electrodes 114 or another electrode originating from the Flexible Printed Circuit Board (FPCB) 110, may extend from the flexible diaphanous sheet 116 to make direct contact with the user's skin. The tactile engagement of the electrodes with the user's skin initiates the activation of the plurality of tensed electrotherapy elements, thereby administering therapeutic treatment to the user.

FIG. 3 illustrates an exploded view of the detachable controller 100B, in accordance with an embodiment of the present invention. In several embodiments of the present invention, the hollow housing 119 of the detachable controller 100B is of square shape, although it is recognized by those skilled in the art that alternate configurations/shapes may be conceivable. In several embodiments of the present invention, the rechargeable battery 120 incorporated in the detachable controller 100B encompasses various types, including but not limited to nickel-cadmium (NiCd), nickel-metal hydride (NiMH), and lithium-ion (Li-ion). Furthermore, the voltage applied to the rechargeable battery 120 falls within the range of 1.2 volts to 3.7 volts. Further, the user interface 124 enables the user to operate various functions, such as switching the lights or other different modes of operation.

In several embodiments of the present invention, the at least one pair of secondary electrodes 122 is crafted over a bottom portion 121 of the hollow housing 119. The at least one pair of secondary electrodes 122 encompassing both a positive terminal and a negative terminal. The positive terminal and the negative terminal are integral components within the detachable controller 100B and are electrically linked to the rechargeable battery 120.

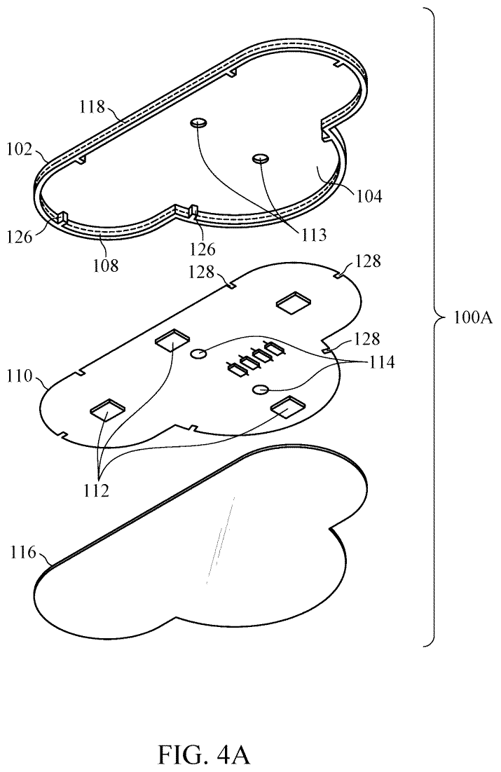

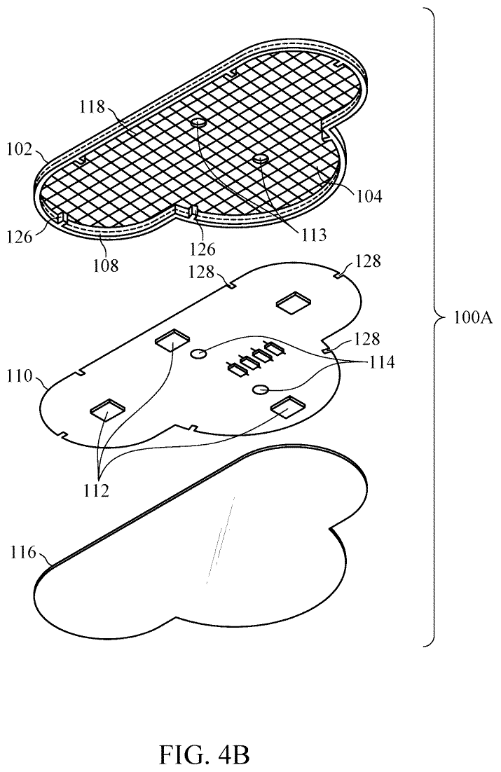

FIG. 4A illustrates an exploded view of the patch 100A designed with a deformable element 118, in accordance with an embodiment of the present invention. In several embodiments of the present invention, the flexible casing 102 of the light therapy device 100 is intrinsically integrated with at least one deformable element 118. The at least one deformable element 118 is preferably crafted from a metallic material comprised of ductile properties, ensuring the deformation of the patch 100A during the application of the patch 100A over the user's body. The choice of a ductile material prevents the at least one deformable element 118 from failing due to deformation. Although, it is recognized that alternate materials may be conceivable for those skilled in the art.

The at least one deformable element 118 in the configuration serves the crucial function of establishing a secure grip between the patch 100A and the user's skin. The secure grip ensures that there is no vacant space left at the periphery of the patch 100A during usage of the patch 100A over the user's body portion. The incorporation of the at least one deformable element 118 enhances the adherence of the light therapy device 100 to the contours of the user's body, optimizing the therapeutic effectiveness.

FIG. 4B illustrates an exploded view of the patch 100A designed with a deformable element 118 in another configuration, in accordance with an embodiment of the present invention. In several embodiments of the present invention, the deformable element 118 may be at the periphery of the flexible casing 102 or, at the back side of the flexible casing 102, or at both the periphery and the back side of the flexible casing 102, (as shown in FIG. 4B). The function of the deformable element 118 is same as described above under the description of FIG. 4A.

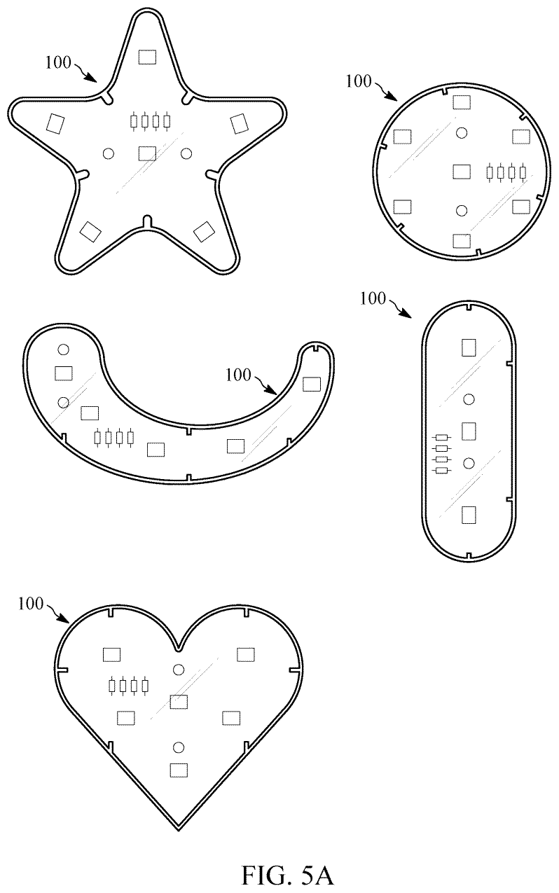

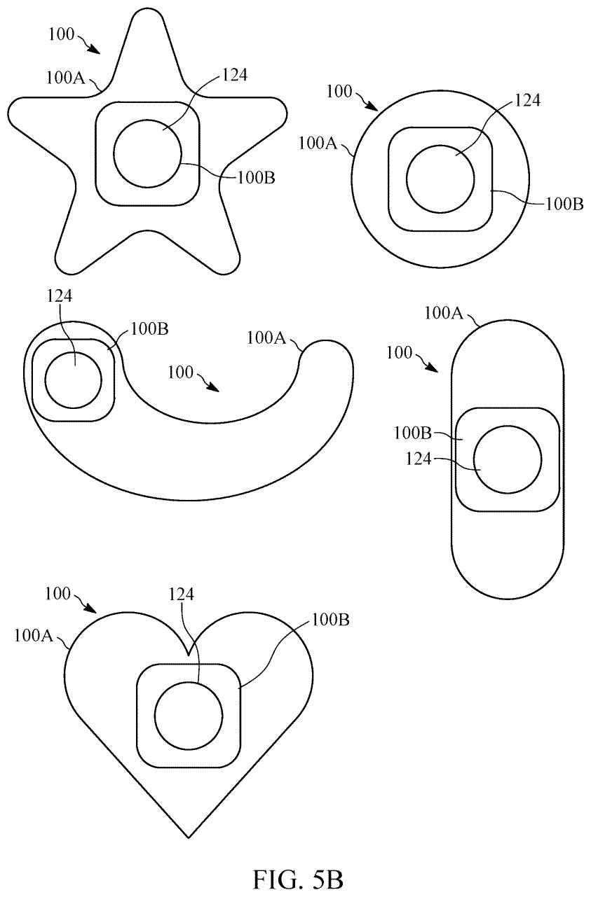

FIG. 5A and FIG. 5B illustrate a bottom view of the light therapy device 100 in various shapes and a top view of the light therapy device 100 in various shapes, respectively, in accordance with an embodiment of the present invention. In numerous embodiments disclosed in the present invention, FIG. 5A and FIG. 5B illustrate a light therapy device 100 featuring various shapes such as circular, moon, and heart, among others, although it is recognized that alternate configurations may be conceivable by those skilled in the art. Despite the diversity in shapes, all iterations of the light therapy device 100 shares a common purpose: to administer therapeutic sessions to users, catering to wound healing and pain relief.

The invention, as described above, offers several advantages. For instance, the light therapy device is very simple in design and construction. Further, the light therapy device uses commonly available materials. The simplicity in design and construction, and the use of commonly available materials allow the light therapy device to be mass-produced with minimal capital expenditure, and to be made available in the market at significantly lower prices. Also, the light therapy device can be reused several times and is, therefore, cost-effective for the end user and minimizes waste generation.

Various modifications to these embodiments are apparent to those skilled in the art, from the description and the accompanying drawings. The principles associated with the various embodiments described herein may be applied to other embodiments. Therefore, the description is not intended to be limited to the embodiments shown along with the accompanying drawings but is to provide the broadest scope consistent with the principles and the novel and inventive features disclosed or suggested herein. Accordingly, the invention is anticipated to hold on to all other such alternatives, modifications, and variations that fall within the scope of the present invention and appended claims.

1. A light therapy device comprising a patch and a detachable controller, the patch comprising:
a flexible casing, the flexible casing comprises a front portion, a back portion, and a peripheral wall, the peripheral wall crafted over the periphery of the front portion;
a Flexible Printed Circuit Board (FPCB) installed over the front portion, the Flexible Printed Circuit Board comprising a plurality of stimulation elements; and
a flexible diaphanous sheet arranged over the Flexible Printed Circuit Board (FPCB),
wherein the detachable controller is attached to the back portion of the flexible casing, and
wherein at least one deformable element is inbuilt within the flexible casing.

2. The light therapy device claimed in claim 1, wherein at least one pair of through-holes is formed over the back portion of the flexible casing.

3. The light therapy device claimed in claim 1, wherein the flexible casing is constructed from opaque material to obstruct the passage of light through the flexible casing.

4. The light therapy device claimed in claim 1, wherein the Flexible Printed Circuit Board (FPCB) comprises at least one pair of primary electrodes passing through the at least one pair of through-holes.

5. The light therapy device claimed in claim 1, wherein the detachable controller comprises:
a hollow housing includes at least one pair of secondary electrodes, the at least one pair of secondary electrodes magnetically attached with the at least one pair of primary electrodes;
a rechargeable battery is installed within the controller to power the light therapy device; and
a user interface, the user interface is crafted over the hollow housing, the user interface is electrically connected to the rechargeable battery to operate the light therapy device.

6. The detachable controller claimed in claim 5, wherein the user interface situated on the side walls of the controller includes but is not limited to a push button, and a touch panel.

7. The light therapy device claimed in claim 1, wherein the shape of the patch includes but is not limited to a star shape, a moon shape, a hexagon shape, a heptagon shape, an octagon shape, and a heart shape.

8. The light therapy device claimed in claim 1, wherein the plurality of stimulation elements is selected from a group consisting of irradiation sources, heating elements, cooling elements, vibration elements, ultrasonic wave generators, electrodes, and combinations thereof.

9. The irradiation sources claimed in claim 8, wherein the irradiation sources are configured to emit electromagnetic radiation in a wavelength range of 300 nm to 1200 nm.

10. The light therapy device claimed in claim 1, wherein the at least one deformable element is made with a metallic material.

11. The light therapy device claimed in claim 1, wherein an insulation separates the Flexible Printed Circuit Board (FPCB) and the primary electrodes from the deformable element.

12. The light therapy device claimed in claim 1, wherein the light therapy device is detachably attached to a body portion of the user using an adhesive agent to enable the user to reuse the light therapy device multiple times.

13. The light therapy device claimed in claim 1, wherein the adhesive agent includes but is not limited to a glue, a gel, and a double-sided tape.

14. A light therapy device comprising a patch and a detachable controller, the patch further comprising:
a flexible casing, the flexible casing comprises a front portion, a back portion, and a peripheral wall, the peripheral wall crafted over the periphery of the front portion;
a plurality of protrusions of a predetermined length formulated inside the flexible casing;
a Flexible Printed Circuit Board (FPCB) installed over the front portion, the Flexible Printed Circuit Board comprising a plurality of stimulation elements;
a flexible diaphanous sheet arranged over the Flexible Printed Circuit Board (FPCB); and
a plurality of cuts crafted over the periphery of the Flexible Printed Circuit Board (FPCB) at a predetermined distance between each of the cuts,
wherein the number of said cuts are equivalent to the number of said protrusions,
wherein the detachable controller is attached to the back portion of the flexible casing, and
wherein each of the cuts is engaged with the corresponding protrusion.

15. The light therapy device claimed in claim 14, wherein at least one pair of through-holes is formed over the back portion of the flexible casing.

16. The light therapy device claimed in claim 14, wherein the Flexible Printed Circuit Board (FPCB) comprises at least one pair of primary electrodes passing through the at least one pair of through-holes.

17. The light therapy device claimed in claim 14, wherein the shape of the patch includes but is not limited to a star shape, a moon shape, a hexagon shape, a heptagon shape, an octagon shape, and a heart shape.

18. The light therapy device claimed in claim 14, wherein the plurality of stimulation elements is selected from a group consisting of irradiation sources, heating elements, cooling elements, vibration elements, ultrasonic wave generators, electrodes, and combinations thereof.

19. The irradiation sources claimed in claim 18, wherein the irradiation sources are configured to emit electromagnetic radiation in a wavelength range of 300 nm to 1200 nm.

20. The light therapy device claimed in claim 14, wherein the at least one deformable element is made with a metallic material.
